(19) United States
(12) Patent Application Publication
(10) Pub. No.: US 2009/0117654 A1
(43) Pub. Date: May 7, 2009

(54) MEDIUM FOR CULTURING HEMATOPOIETIC CELLS AND A METHOD OF CULTURING HEMATOPOIETIC CELLS

(75) Inventors: Byung-Chul Kim, Gyeonggi-do (KR); Sook-Young Kim, Gyeonggi-do (KR); Sung-Woo Hong, Gyeonggi-do (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si (KR)

(21) Appl. No.: 12/300,440

(22) PCT Filed: Nov. 8, 2006
(86) PCT No.: PCT/KR06/04656

§ 371 Date: Nov. 11, 2008

(30) Foreign Application Priority Data
May 11, 2006 (KR) 10-2006-0042386

(51) Int. Cl. C12N 5/02

(52) U.S. Cl. 435/383; 435/404

(57) ABSTRACT

Provided are a medium for culturing hematopoietic cells including lysyl oxidase inhibitor and a method of culturing hematopoietic cells using the medium.

### CROSS-REFERENCE TO RELATED PATENT APPLICATION

This application claims the benefit of Korean Patent Application No. 10-2006-0042386, 11 May, 2006, in the Korean Intellectual Property Office, the disclosure of which is incorporated herein in its entirety by reference.

### BACKGROUND OF THE INVENTION

1. Field of the Invention

The present invention relates to a medium for culturing hematopoietic cells and a method of culturing hematopoietic cells.

2. Description of the Related Art

Bone marrow cells, blood cells, and cord blood cells are known as sources for hematopoietic cell transplantation. Those cells contain hematopoietic stem cells which can be differentiated into blood cells.

Bone marrow is a complex and dynamic system consisting of hematopoietic stem cells, stromal cells, and extracellular matrix. Hematopoietic stem cells in the bone marrow proliferate, differentiate, and develop into several types of blood cells including erythrocytes and leucocytes. Interaction between hematopoietic stem cells and stromal cells has been known as a key process for maintenance of the stem cell properties. For in vitro cell culture, an adsorbent stromal cell layer on the surface of culture dishes has been used for the hematopoietic stem cells.

Bone marrow stromal cells are a heterogeneous population of cells that are classified by their morphology and function. In cell culture, they have a characteristic spindle-shaped morphology and secrete growth factors and components that form extracellular matrix. For replacing the stromal cells, serum-free media has been developed to provide the cell microenvironment and individual growth factors such as stem cell factor (SCF), interleukin-3 (IL-3), interleukin-6 (IL-6), thrombopoietin (TPO), and fms-like tyrosine kinase-3 ligand (FL).

Protein-lysine 6-oxidase (hereinafter referred to as lysyl oxidase) is a cuproenzyme which is essential to stabilizations in extracellular matrix, and more particularly to an enzymatic cross-linking of collagen and elastin. Cell fibronectin forms a strong bind with lysyl oxidase, and has been known to be essential to activate protein decomposition of lysyl oxidase (J. Biol. Chem. 2005 Jul. 1; 280(26):24690-7).

Hematopoietic cell transplantation is one of prospective treatments for various diseases related to hematopoietic stem cells. Transplanted hematopoietic cells replace hematopoietic cells damaged by an intrinsic disease such as anemia or hematopoietic cells damaged by chemotherapy or radiotherapy. The transplantation may be an autologous transplantation, that is the donor and the recipient are the same. In addition, a patient can receive bone marrow from a donor whose tissue type is almost identical to the patient. However, conditions for culturing hematopoietic cells which are transplantable and available for various gene treatments have not been optimized. Accordingly, there is still a need to develop mediums for and methods of effectively culturing hematopoietic cells.

The present inventors have worked on the method of culturing hematopoietic cells and developed the present invention in which a lysyl oxidase inhibitor including β-aminopropionitrile (BAPN) promotes the proliferation of bone marrow cells mostly consisting of hematopoietic cells.

### SUMMARY OF THE INVENTION

The present invention provides a medium for culturing hematopoietic cells to effectively proliferate hematopoietic cells.

The present invention also provides a method of culturing hematopoietic cells using the medium.

According to an aspect of the present invention, there is provided a medium for culturing hematopoietic cells including a lysyl oxidase inhibitor.

According to another aspect of the present invention, there is provided a method of culturing hematopoietic cells including: introducing the hematopoietic cells into a culture vessel including the medium for culturing hematopoietic cells; and culturing the hematopoietic cells.

### BRIEF DESCRIPTION OF THE DRAWINGS

The above and other features and advantages of the present invention will become more apparent by describing in detail exemplary embodiments thereof with reference to the attached drawings in which:

FIG. 1 is a graph illustrating effects of BAPN on proliferation of bone marrow cells; and

FIG. 2 is a graph illustrating effects of BAPN on proliferation of bone marrow cells in a medium including IL-3.

### DETAILED DESCRIPTION OF THE INVENTION

Hereinafter, the present invention will now be described more fully with reference to the accompanying drawings, in which exemplary embodiments of the invention are shown. The invention may, however, be embodied in many different forms and should not be construed as being limited to the embodiments set forth herein; rather, these embodiments are provided so that this disclosure will be thorough and complete, and will fully convey the concept of the invention to those skilled in the art.

According to an embodiment of the present invention, there is provided a medium for culturing hematopoietic cells including a lysyl oxidase inhibitor.

In the medium, the lysyl oxidase inhibitor may be an inhibitor which is bound to the active site of lysyl oxidase and directly inhibits the activity of the lysyl oxidase or an inhibitor which is bound to a non-active site or another regulatory region and inhibits the activity of the lysyl oxidase. Examples of directly inhibiting the activity of lysyl oxidase may include (a) a primary amine that reacts with a carbonyl group of the lysyl oxidase, and more particularly a material that binds to the carbonyl group of the lysyl oxidase and produces a stabilized product by resonance, for example, ethylenediamine, hydrazine, phenylhydrazine and its derivatives, semicarbazide, urea derivatives, aminonitrile such as β-aminopropionitrile, 2-nitroethylamine, a saturated or unsaturated haloamine such as 2-bromoethylamine, 2-chloroethylamine, 2-trifluoroethylamine, 3-bromopropylamine and p-haloethylamine, and selenohomocysteine lactone, b) copper chelating agent, and c) an antibody binding to the active site of the lysyl oxidase. Examples of indirectly inhibiting the activity of lysyl oxidase may include (a) a material that inhibits aldehyde derivatives generated by oxidative deamination of lysyl or hydroxy lysyl moiety by lysyl oxidase, for example thiolamine, D-penicillamine, 2-amino-5-mercapto-5-methylhexanoic acid, D-2-amino-3-methyl-3-((2-acetamidoethyl)(butanoic acid), p-2-amino-3-methyl-3-((2-aminoethyl)dithio)butanoic acid, sodium-4-((p-1-dimethyl-2-amino-2-carboxyethyl)dithio)butane sulphinate, 2-acetamidoethyl-2-acetamidoethanethiol sulphanate, and sodium-4-mercaptobutane sulphinate trihydrate, and b) a material inhibiting biosynthesis of lysyl oxidase such as antisense. Preferably, the lysyl oxidase inhibitor is β-aminopropionitrile. The concentration of β-aminopropionitrile may be from 10 to 200 μg/ml.

The medium for culturing hematopoietic cells of an embodiment of the present invention may be prepared by adding a lysyl oxidase inhibitor to a conventional medium for culturing hematopoietic cells to improve proliferation of the hematopoietic cells. The lysyl oxidase is a copper dependant enzyme, and involved in cross-linking collagen and elastin, which are the components of extracellular matrix. It is known that copper deficiency is an important factor in the proliferation of bone marrow cells and hematopoietic stem cells, but it is difficult to target copper itself due to its various physiological functions. For example, it is considered that β-aminopropionitrile can reversibly inhibit lysyl oxidase, accumulate extractable collagen containing a hydroxyl lysine cross-linking precursor, and inhibit collagen cross-linking, e.g., dehydroxylysinonorleucine (DHLNL) formation, to decrease integration, but the present invention is not limited thereto. Generally, it is known that such a decrease in integration may cause growth disorders in normal tissues, and lysyl oxidase is expressed and activated in cardiovascular diseases and cancers differently from under normal conditions. Meanwhile, it was reported that an inhibition of lysyl oxidase activity may prevent or delay dedifferentiation of chondrocytes with reference to cell culture (Osteoarthritis Cartilage 2005 13:120-8), but research on culturing hematopoietic cells in vitro has not been reported.

A conventional medium for culturing hematopoietic cells is a basal medium further including specific ingredients required for culturing hematopoietic cells. The basal medium may be a serum-free medium or a serum-containing medium. The serum-free medium may be an Iscove's MDM (IMDM) including 10 μg/ml of recombinant human insulin, 100 μg/ml of bovine serum albumin (BSA), 200 μg/ml of human transferrin, 0.1 mM of 2-mercaptoethanol, and 2 mM of L-glutamine, but is not limited thereto. The basal medium may be commercially available, and the composition thereof may be obvious to those of skilled in the art. For example, the composition of IMDM is disclosed in U.S. Pat. No. 5,945,337. The basal medium is prepared to provide i) inorganic salts (e.g., potassium, calcium, phosphate, etc.) to maintain cell osmolality and mineral requirements, ii) essential amino acids which are not synthesized in intrinsic metabolism of cells and are essential for cell growth, iii) carbon sources such as glucose which is used for cell energy metabolism, and iv) various vitamins and cofactors which can be required in cell growth such as riboflavin, nicotinamide, folic acid, choline, and biotin. Glutamine is one of essential amino acids that can be added to the medium of an embodiment of the present invention in an effective amount. The concentration of glutamine may be from 100 to 500 μg/ml, preferably from 125 to 375 μg/ml, and more preferably from 150 to 300 μg/ml. Glutamine is sometimes added to the medium immediately before the use of the medium due to its instability. A buffer which maintains the pH of the medium during the cell metabolism may be added to the basal medium. In general, the buffer may be a bicarbonate or HEPES. The pH of the basal medium is generally in the range of 6.8 to 7.2.

The medium for culturing hematopoietic cells of an embodiment of the present invention may further include cytokine which is required for the growth of hematopoietic cells in the basal medium as described above. For example, at least one cytokine selected from the group consisting of 10 to 200 ng/ml of stem cell factor (SCF), 2 to 100 ng/ml of interleukin-3 (IL-3), 2 to 100 ng/ml of interleukin-6 (IL-6), 10 to 200 ng/ml of thrombopoietin (TPO), and 10 to 200 ng/ml of fms-like tyrosine kinase-3 ligand (FL) is included in the basal medium.

Examples of the medium for culturing hematopoietic cells of an embodiment of the present invention may include lysyl oxidase in the basal medium and further include 10 to 200 ng/ml of SCF, 10 to 200 ng/ml of TPO, 10 to 200 ng/ml of FL, 1 to 20 μg/ml of insulin, 50 to 150 μg/ml of bovine serum albumin, 100 to 300 μg/ml of human transferrin, 0.05 to 0.5 mM of 2-mercaptoethanol, and 1 to 5 mM of glutamine. Preferably, the medium may further include 2 to 100 ng/ml of IL-6.

The medium may further include IL-3. The concentration of IL-3 may be from 2 to 100 ng/ml.

The medium may further include G-CSF. The concentration of G-CSF may be from 20 to 100 ng/ml.

In the present embodiment, the term “hematopoietic cells” indicates cells capable of reconstituting hematopoietic function of a recipient after hematopoietic cell transplantation. The hematopoietic cells can be obtained from human bone marrow cells, blood cells, and cord blood cells. Accordingly, the term “hematopoietic cells” in the present embodiment is used to indicate a population of cells containing hematopoietic cells such as bone marrow cells, blood cells, and cord blood cells. Here, the term “bone marrow cells” indicates bone marrow derived cells which consist of mostly hematopoietic stem cells and hemocytes differentiated from the hematopoietic stem cells, and partially bone marrow stromal cells involved in differentiation and proliferation of the hematopoietic cells. Bone marrow cells may be commercially available (e.g., Stem Cell Technology Co. Ltd. Canada) or may be obtained from a person. Bone marrow is a complex and dynamic organic system consisting of hematopoietic stem cells, stromal cells, and extracellular matrix. Multipotent stem cells in the bone marrow proliferate, differentiate, and develop into several types of cells including erythrocytes and leucocytes. As a result of culturing cells, an adsorbent stromal cell layer should be established before the hematopoietic cells grow and differentiate. Accordingly, in order to culture hematopoietic cells in vitro without stromal cells, cytokines such as SCF, TPO, IL-3, IL-6 and FL are required.

Thus, the medium for culturing hematopoietic cells of an embodiment of the present invention may include an ingredient such as cytokine which is known to promote the proliferation of the hematopoietic cells.

According to an embodiment of the present invention, there is provided a method of culturing hematopoietic cells including: introducing the hematopoietic cells into a culture vessel including the medium for culturing hematopoietic cells according to an embodiment of the present invention; and culturing the hematopoietic cells.

The method of culturing hematopoietic cells may include introducing the hematopoietic cells into a culture vessel including a medium for culturing hematopoietic cells containing a lysyl oxidase inhibitor.

In the method, the terms “hematopoietic cells”, “lysyl oxidase inhibitor”, and “medium for culturing hematopoietic cells” are described above. Hematopoietic cells may be commercially available, or obtained from a patient. The lysyl oxidase inhibitor that is used in the method may vary, but preferably is β-aminopropionitrile.

In the method of the present embodiment, the culture vessel may be any vessel that is commonly used in the art, and examples of the culture vessel may include a flask, a plate having various numbers of wells, a roller bottle, and a cell reaction vessel, etc.

In addition, the hematopoietic cells may be introduced into the culture vessel by any method that is commonly used in the art. For example, the hematopoietic cells may be inoculated into the medium using a pipette, or the hematopoietic cells may be introduced into the culture vessel by a decantation or using a pipe.

The method of an embodiment of the present invention also includes culturing hematopoietic cells. The culturing of hematopoietic cells may be performed under any conditions that are known in the art. For example, the hematopoietic cells may be cultured in a 37° C. incubator with 100% humidity and an atmosphere of CO₂, with half of the medium being exchanged as frequently as every week, but the conditions for culturing the hematopoietic cells are not limited thereto.

In the method according to an embodiment of the present invention, the culture vessel may be coated with extracellular matrix. The extracellular matrix is known to promote the growth of the hematopoietic cells. Preferably, the extracellular matrix may include at least one material selected from the group consisting of fibronectin and collagen, but is not limited thereto.

Hereinafter, the present invention will be described in greater detail with reference to the following examples. The following examples are for illustrative purposes only and are not intended to limit the scope of the invention.

### EXAMPLES

### Example 1

### Effects of BAPN on Proliferation of Bone Marrow Cells in Serum-Free Basal Medium

Effects of β-aminopropionitrile (hereinafter referred to as BAPN) on proliferation of bone marrow cells in serum-free medium were inspected. The bone marrow cells used in Example 1 mostly consists of hematopoietic cells and is a population of cells including hematopoietic cells.

First, proliferations of bone marrow cells were inspected in a serum-free medium in the presence and absence of fibronectin and collagen IV which are extracellular matrix that is known to influence proliferation of bone marrow cells.

A bone marrow monocyte (Catalog number ARM07F) was purchased from Stem Cell Technology Co., Ltd. The bone marrow mononuclear cells was obtained from a normal person, and CD34+ cells accounted for 1.6% of the cells. The bone marrow cells were maintained in a maintenance medium. The maintenance medium was a IMDM including 50 ng/ml of SCF, 50 ng/ml of IL-6, 50 ng/ml of TPO, 50 ng/ml of FL, 10 μg/ml of recombinant human insulin, 100 μg/ml of bovine serum albumin (BSA), 200 μg/ml of human transferrin, 0.1 mM of 2-mercaptoethanol, and 2 mM of L-glutamine.

100 μl of the basal medium, and 100 μl of the basal medium with 100 μg/ml of BAPN were placed in each well of a 24 well plate, bone marrow cells were inoculated onto the media in the concentration of 1×10⁵ cell/ml, and cultured for two weeks. The medium was exchanged every week. After the two week culture, adhesive cells were detached from the surface through trypsin treatment and the final cell concentration including floating cells was measured using a hemacytometer. In addition, the bottom of the 24 well plate was coated with collagen IV or fibronectin, and the effects of collagen IV or fibronectin were measured at the same time. The coating materials were dissolved to the concentration of from 5 to 50 μg/ml, the mixture was poured into the 24 wells to the amount of from 1 to 10 μg/cm² and reacted for one hour at room temperature.

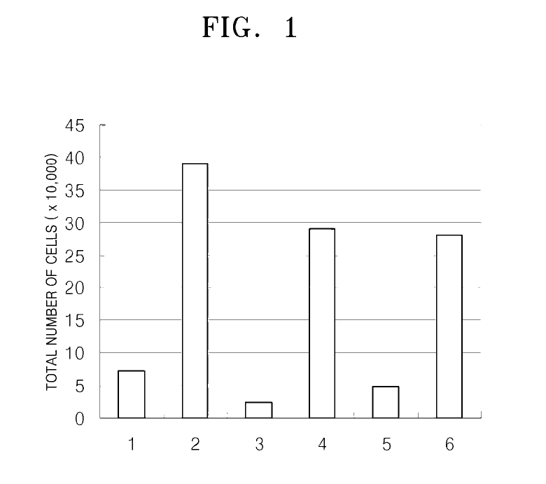

FIG. 1 is a graph illustrating effects of BAPN on proliferation of bone marrow cells. As illustrated in FIG. 1, the bone marrow cells were proliferated 5.4 times more in the medium including BAPN than the control medium. When a 24 well plate that was coated with fibronectin was used, the bone marrow cells were proliferated 12.1 times more in the medium including BAPN than the control medium. When a 24 well plate that was coated with collagen was used, the bone marrow cells were proliferated 5.1 times more in the medium including BAPN than the control medium. In FIG. 1, the media are: 1: control medium, 2: a medium in which BAPN is added to the control medium, 3: control medium in the fibronectin-coated culture vessel, 4: a medium in which BAPN is added to the control medium in the fibronectin-coated culture vessel, 5: control medium in the collagen-coated culture vessel, and 6: a medium in which BAPN is added to the control medium in the collagen-coated culture vessel.

The results of FIG. 1 showed that BAPN promoted the proliferation of bone marrow cells whether or not the substrate of the culture vessel was coated with extracellular matrix.

### Example 2

### Effects of BAPN on Proliferation of Bone Marrow Cells in Serum-Free Basal Medium Including IL-3

Effects of BAPN on proliferation of bone marrow cells in serum-free basal medium including IL-3 which induces divisions of hematopoietic stem cells were inspected.

A bone marrow monocyte (Catalog number ARM07F) was purchased from Stem Cell Technology Co., Ltd. The bone marrow monocyte was obtained from a normal person, and CD34+ cells accounted for 1.6% of the total monocytes. The bone marrow cells were maintained in a maintaining medium. The maintaining medium was a IMDM including 50 ng/ml of SCF, 50 ng/ml of IL-6, 50 ng/ml of TPO, 50 ng/ml of FL, 10 μg/ml of recombinant human insulin, 100 μg/ml of bovine serum albumin (BSA), 200 μg/ml of human transferrin, 0.1 mM of 2-mercaptoethanol, and 2 mM of L-glutamine.

100 μl of the basal medium with 20 μg/ml of IL-3, and 100 μl of the basal medium with 20 μg/ml of IL-3 and 100 μg/ml of BAPN were placed in each well of a 24 well plate, bone marrow cells were inoculated onto the media in the concentration of 1×10⁵ cell/ml, and cultured for two weeks. The medium was exchanged every week. After the 2 week culture, adhesive cells were detached from the surface through trypsin treatment and the final cell concentration including floating cells was measured using a hemacytometer. In addition, the bottom of the 24 well plate was coated with collagen IV or fibronectin, and the effects of collagen IV or fibronectin were measured at the same time. In the coating, coating materials were dissolved to the concentration of from 5 to 50 μg/ml, the mixture was poured into the 24 wells to the amount of from 1 to 10 μg/cm² and reacted for one hour at room temperature.

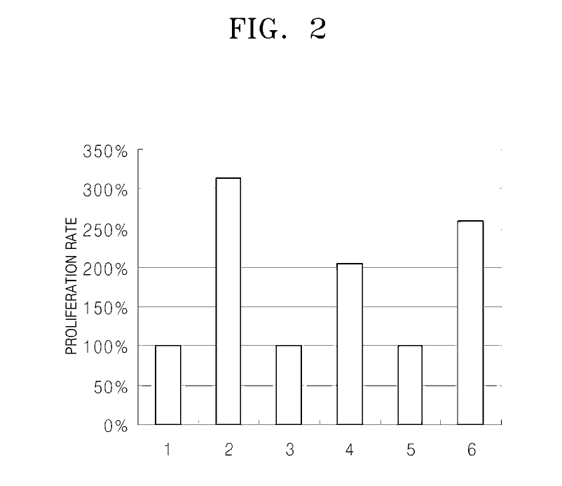

FIG. 2 is a graph illustrating effects of BAPN on proliferation of bone marrow cells in a medium including IL-3. As illustrated in FIG. 2, the bone marrow cells were proliferated 3.1 times more in the medium including IL-3 and BAPN than the control medium including IL-3. When a 24 well plate that was coated with fibronectin was used, the bone marrow cells were proliferated 2.0 times more in the medium including IL-3 and BAPN than the control medium including IL-3. When a 24 well plate that was coated with collagen was used, the bone marrow cells were proliferated 2.6 times more in the medium including IL-3 and BAPN than the control medium including IL-3. In FIG. 2, the media are: 1: a medium in which IL-3 is added to the control medium, 2: a medium in which IL-3 and BAPN are added to the control medium, 3: a medium in which IL-3 is added to the control medium in the fibronectin-coated culture vessel, 4: a medium in which IL-3 and BAPN are added to the control medium in the fibronectin-coated culture vessel, 5: a medium in which IL-3 is added to the control medium in the collagen-coated culture vessel, and 6: a medium in which IL-3 and BAPN are added to the control medium in the collagen-coated culture vessel.

The results of FIG. 2 showed that BAPN promoted the proliferation of bone marrow cells whether or not the substrate of the culture vessel was coated with extracellular matrix and whether or not in the presence of cytokine which is required for the growth of hematopoietic cells.

The medium for culturing hematopoietic cells of the present invention promotes the proliferation of the hematopoietic cells and can be used to effectively proliferate the hematopoietic cells.

According to the method of culturing hematopoietic cells of the present invention, the hematopoietic cells can be effectively proliferated.

Therefore, the hematopoietic cells obtained using the medium for culturing hematopoietic cells and the method of culturing hematopoietic cells can be used in various fields. The hematopoietic stem cells included in the hematopoietic cells can be differentiated into complete blood cells under appropriate conditions. In addition, the proliferated hematopoietic cells can be effectively used for cell treatments, tissue transplantations, medicine developments, and anticancer treatments.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A medium for culturing hematopoietic cells comprising a lysyl oxidase inhibitor.

2. The medium of claim 1, wherein the lysyl oxidase inhibitor is β-aminopropionitrile.

3. The medium of claim 2, wherein the concentration of the β-aminopropionitrile is from 10 to 200 μg/ml.

4. The medium of claim 1, wherein the hematopoietic cells are included in bone marrow cells, blood cells or cord blood cells.

5. The medium of claim 4, wherein the hematopoietic cells are included in the bone marrow cells, and the medium further comprises 10 to 200 ng/ml of stem cell factor (SCF), 10 to 200 ng/ml of thrombopoietin (TPO), 10 to 200 ng/ml of fins-like tyrosine kinase-3 ligand (FL), 1 to 20 μg/ml of insulin, 50 to 150 μg/ml of bovine serum albumin, 100 to 300 μg/ml of human transferrin, 0.05 to 0.5 mM of 2-mercaptoethanol, and 1 to 5 mM of glutamine.

6. The medium of claim 5, further comprising 2 to 100 ng/ml of IL-6.

7. The medium of claim 1, being a serum-free medium.

8. The medium of claim 1, wherein the basal medium is an Iscove's MDM (IMDM).

9. The medium of claim 1, further comprising IL-3.

10. The medium of claim 9, wherein the concentration of IL-3 is from 2 to 100 ng/ml.

11. A method of culturing hematopoietic cells comprising:
introducing the hematopoietic cells into a culture vessel comprising a medium for culturing hematopoietic cells according to claim 1; and
culturing the hematopoietic cells.

12. The method of claim 11, wherein the culture vessel is coated with extracellular matrix.

13. The method of claim 11, wherein the extracellular matrix comprises at least one selected from the group consisting of fibronectin and collagen.

14. The method of claim 11, wherein the lysyl oxidase inhibitor is β-aminopropionitrile.

15. The method of claim 14, wherein the concentration of the β-aminopropionitrile is from 10 to 200 μg/ml.

16. The method of claim 11, wherein the hematopoietic cells are included in bone marrow cells, blood cells or cord blood cells.

17. The method of claim 16, wherein the hematopoietic cells are included in the bone marrow cells, and the medium further comprises 10 to 200 ng/ml of stem cell factor (SCF), 10 to 200 ng/ml of thrombopoietin (TPO), 10 to 200 ng/ml of fins-like tyrosine kinase-3 ligand (FL), 1 to 20 μg/ml of insulin, 50 to 150 μg/ml of bovine serum albumin, 100 to 300 μg/ml of human transferrin, 0.05 to 0.5 mM of 2-mercaptoethanol, and 1 to 5 mM of glutamine.

18. The method of claim 17, wherein the medium further comprising 2 to 100 ng/ml of IL-6.

19. The method of claim 11, wherein the medium being a serum-free medium.

20. The method of claim 11, wherein the basal medium is an Iscove's MDM (IMDM).

21. The method of claim 11, wherein the medium further comprising IL-3.

22. The method of claim 21, wherein the concentration of IL-3 is from 2 to 100 ng/ml.
